(19) United States
(12) Patent Application Publication
(10) Pub. No.: US 2004/0023210 A1
(43) Pub. Date: Feb. 5, 2004

(54) Diagnostic kit for invitro detection of hepatitis C

(76) Inventors: Lalit Mahajan, New Delhi (IN); Renu Malhotra, New Delhi (IN); Mamta Tiwari, New Delhi (IN)

Correspondence Address: LADAS & PARRY, 26 WEST 61ST STREET, NEW YORK, NY, 10023, US

(21) Appl. No.: 10/311,056

(22) PCT Filed: Jun. 14, 2001
(86) PCT No.: PCT/IN01/00115

(30) Foreign Application Priority Data
Jun. 14, 2000 (IN) 593/DEL/2000

(51) Int. Cl. C12Q 1/70

(52) U.S. Cl. 435/5

(57) ABSTRACT

A rapid, visual, sensitive and qualitative in vitro diagnostic kit for the detection of antibodies of Hepatitis C Virus in human serum and plasma comprising a testing device, a buffer solution and a Protein A-Conjugate, wherein said testing device comprises a base, an absorbent pad positioned on the base, an immunofilteration membrane mounted over said absorbent pad disposed on the said base, a top cover removably attached to said base having a central hole conforming to the circumference of said immunofilteration membrane provided with two test dots and one built in quality control dot within the circumference of said immunofilteration membrane to render 100% sensitivity and 98.9% specificity of the sample under test.

The present invention relates to a diagnostic kit for in-vitro detection of liver diseases such as Hepatitis C Virus antibodies in the human plasma and serum.

More specifically, the subject invention relates to a novel, diagnostic kit herein after referred as fourth generation diagnostic kit for the detection of antigens for Hepatitis C Virus in the human serum and plasma.

The diagnostic kit of the subject invention comprises a test device, a buffer solution and a Protein-A Conjugate solution.

### PRIOR ART

The present HCV test kits as available for the detection of HCV antibodies in human serum or plasma, lacks in providing desired specificity and sensitivity, thus occasionally not able to detect the HCV at the early stages.

Hepatitis C Virus was identified in 1989 as the main aetiological agent of non-A, non-B Hepatitis accounting for greater than 90% of post-transfusion hepatitis case. Hepatitis C Virus is a spherical virus of about 30-60 mm in diameter with single positive standard RNA and is related to the family flaviviridae. It is considered to be the major cause of acute chronic hepatitis, liver cirrhosis and hepatocellular carcinoma throughout the world.

HCV infection is a major cause of chronic liver disease and hepatocellular carcinoma. It is therefore, important to make a precise and accurate diagnosis, and to follow and treat hepatitis C infection.

Chronic infection is a major cause of chronic liver disease and hepatocellular carcinoma worldwide. It is therefore necessary to correctly diagnose hepatitis C infection. Such diagnosis necessitated the requirement of sensitive tools including antibody assay and genomic assay.

A series of sensitive and specific tests are available for the diagnosis and evaluation of patients with HCV infection. C100-3 peptide have been used in the first generation enzyme linked immunosorbent assay (EIA-1) to detect anti-HCV. This prototype test for antibodies to HCV was found to be very valuable in the diagnosis and study of the epidemiology of HCV. It however, lacked the sensitivity, specially in the early diagnosis of acute hepatitis. The non-specificity observed in the available tests led to the development of second generation enzyme linked immunosorbent assay (EIA-2) The EIA-2 assay incorporated several recombinant viral proteins or peptides and found to had better sensitivity and specificity then the original EIA-1. The third generation assay for testing of antiHCV was established to overcome the drawbacks found in EIA-2, where peptides of NS5 were added to the peptide used in EIA-2.

The results found in third generation assay were not 100%, hence to achieve 100% results of specificity and sensitivity, the fourth generation assay for testing anti-HCV has been developed to correctly diagnose Hepatitis C infection.

The test for antibodies to Hepatitis C Virus was proved to be highly valuable in the diagnosis of Hepatitis C Virus and study the infection, especially in the early diagnosis of Hepatitis C Virus before transfusion. The diagnosis of Hepatitis C can be easily made by finding elevated serum ALT levels and presence of anti-HCV in serum/plasma.

The recombinant DNA technique have been used to encode the genome of the Hepatitis C Virus. The genome encodes for three structural proteins and several non-structural proteins.

The first and second generation anti Hepatitis C Virus uses synthetic peptide and recombinant viral proteins from non-structural proteins. However, they are associated with both false positive and negative results.

To overcome this drawback, the third generation antibody test using a greater range of antigens from core, NS3, NS4 and NS5 regions of the HCV genome allowing the detection of specific antibodies to multiple viral epitopes and thus providing greater sensitivity and better specificity was developed. The use of these additional antigens results in early detection of antibodies during seroconversion following HCV infection.

The third generation Hepatitis C antibody test using recombinant antigens and HCV synthetic peptides is associated some times with false positive results due to cross reactivity with certain undesirable substances which are not participating in the detection of antibodies but are resulting in giving false positive results.

To overcome this drawback of false results and to achieve 100% reliable results, the fourth generation antibody test using a greater range of antigens from the subtype level of HCV core antigens, HCV NS3 antigens, HCV NS4 antigens, and HCV NS5 antigens regions of the HCV genome allowing the detection of specifics antibodies to multiple viral epitopes is devised, thus providing greater sensitivity and better specificity are used. The use of these selected subtypes results in early detection of antibodies during seroconversion following HCV infection.

A new combination or formulation of antigens have been evolved to overcome the drawbacks existed in the conventionally available diagnostic methods.

This new combination of antigens comprises Non Structural Protein Subtype Three (NS3), Non Structural Protein Subtype Four (NS4), Non Structural Protein Subtype Five (NS5) and Core. Where the core is a structural Protein.

The 4th generation HCV Tridot of subject invention is a rapid, visual, sensitive and qualitative in-vitro diagnostic test for the detection of antibodies to Hepatitis C Virus in human serum and plasma.

The subject invention relates to the fourth generation antibody test for the HCV, utilizing a unique combination of HCV antigens from the subtype level of 2-10 subtypes of putative core, and other non-structural proteins regions of the virus to selectively detect all subtypes of Hepatitis C Virus in human serum/plasma with a high degree of specificity and sensitivity.

The subject invention has resulted in achieving 98.9% of specificity and 100% of sensitivity

The antigens used are chemically treated and unfolded in a desired way to make them more reactive and specific to different epitopes of core and NS3 antibodies thereby minimizing the chances of cross-reactivity and enhancing the specificity.

The superior sensitivity of the test allows for the significantly earlier detection of antibodies during seroconversion following HCV infection, thereby reducing the incidence of post-transfusion hepatitis and providing a safer blood supply.

The subject invention has been developed using HCV antigens representing the immunodominant regions of HCV antigen.

The device of subject invention includes two test points “T₁” and “T₂” and a third point “C” representing “Built in Quality Control Dot”. The function of the control dot is to develop the colour during the test, to confirm proper functioning of the device.

Accordingly, the present invention relates to a rapid, visual, sensitive and qualitative in vitro diagnostic kit for the detection of antibodies of Hepatitis C Virus in human serum and plasma comprising a testing device, a buffer solution and a Protein A Conjugate, wherein said testing device comprises a base, an absorbent pad made up of cellulosic material having a thickness preferably of 2.4 to 2.7 mm positioned on the said base, an immunofilteration membrane made up of cellulosic material having a pore size of 0.8-1.5 micron and diameter of 12 mm having at least three coatings of a homogenous mixture of different HCV recombinant antigens and HCV Peptides and the antihuman IgG solution for the detection of HCV antibodies mounted over said absorbent pad disposed on the said base and the, a top cover fitting tightly and removably attached to said base having a central hole conforming to the circumference of said immunofilteration membrane provided with two test dots and one built in quality control dot within the circumference of said immunofilteration membrane characterized in that the invitro diagnostic kit renders 100% sensitivity and 98.9 specificity of the sample under test.

The subject application may best be understood with reference to the accompanying drawings, which are for illustrative purposes, hence the same should not be construed to restrict the scope of the application.

### BRIEF DESCRIPTION OF THE ACCOMPANYING DRAWINGS

FIG. 1 relates to a test device;

FIG. 2 relates to the membrane used in the test device having plurality of coating of different combinant of antigens on it;

FIGS. 3(a-f) relates to the reactivity or non-reactivity of the test results.

### DETAILED DESCRIPTION OF THE INVENTION

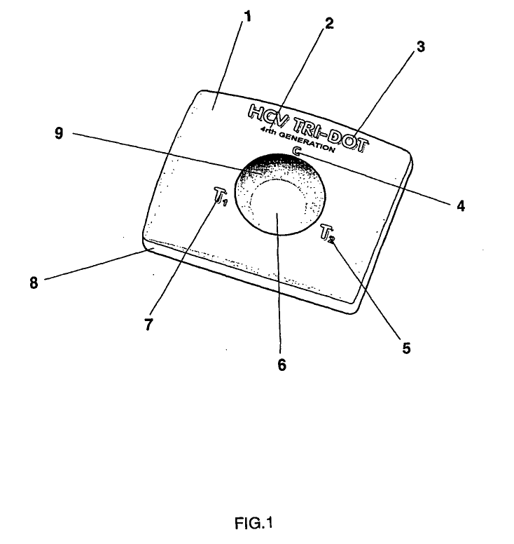

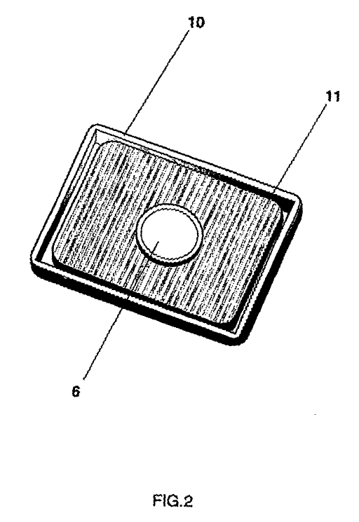

The test device of the subject invention as shown in FIGS. 1 and 2, comprises a membrane (6) having a plurality of coating of different recombinant antigens provided thereon for the detection of HCV, said coated membrane is mounted on a laminated absorbent (11) being disposed in a casing (10) having a central hole provided in the top cover of the said casing for the exposure of said membrane having a plurality of coatings of different recombinant antigens.

The said membrane having pore size of 0.8 micron to 1.5 micron is made up of cellulosic material preferably of nitro cellulose.

The absorbent pad used in the subject applications is also made up of cellulosic material. The thickness of the said absorbent pad is optimized to completely absorb and retain the mixture of buffer, Protein-A conjugate and sample material. The thickness of said absorbent pad preferably is from 2.4 mm to 2.7 mm.

The said circular shaped membrane (6) is cut from the said cellulosic material which is placed on the laminated sheet housed in a casing having the separable top and bottom covers.

The said membrane (6) is coated with plurality of coatings of different recombinant antigens such as Core, NS3, NS4 and NS5.

These antigens can be chemically linked or physically mixed.

The said antigens are mixed taking

| | |
|---|---|
| Core | 40-60 nannogram |
| NS3 | 100-150 nannogram |
| NS4 | 150-200 nannogram |
| NS5 | 150-200 nannogram |

The antigens are mixed in a dispensing agent. The dispensing agent used is buffer solution.

In the process for the preparation of such mixture, the said antigens are mixed in 0.5 microlitre of dispensing agent by stirring in a cyclomixer for 30 seconds to one minute to get a homogenous solution.

0.5 microliter of the said homogenous solution is put on the said immunofilteration membrane.

The composition of the said dispensing media weight/volume is:

| | |
|---|---|
| Disodium Hydrogen Phosphate | 10-100 millimolar; |
| Sodium Dihydrogen Phosphate | 10-100 millimolar; |
| Protein Stabilizer | 0.1-.5% |
| Detergent | 0.02-1% |
| Glycerol | 10-20% |
| and preservative | 0.001-0.15% |
| and the balance being distilled water | |

The detergent used in the subject dispensing media is selected from Dithiothreitol, t-octyl phenoxy poly ethoxy ethanol, Poly oxy ethylene sorbitan monolaurate, 3-(3-Cholamidopropyl) dimethyl ammonio]-1-Propane sulfonate.

The 3-4 drops of buffer solution is dropped on the said circular shaped membrane to activate the same and keep it for 3045 seconds to allow it to be absorbed. 1 drop of sample is then added on the said membrane and keep aside for 5-10 seconds to allow it to be absorbed completely. After that 5-10 drops of the buffer solution is then added on to the said membrane having sample drops deposited on it to remove any unwanted antibodies there from and keeping it aside for 30-45 seconds. 2 drops of Protein A-conjugate is then added and keeping the same for 30 seconds. 3-7 drops of buffer solution is dropped on it to see if there is any colour development at the designated point.

It is important to allow each solution to soak in the test device before adding next solution.

If there is any coloured appearance at the points T1 and T2, then the test is positive and the absence of any coloured appearance indicates the negativity of the test result.

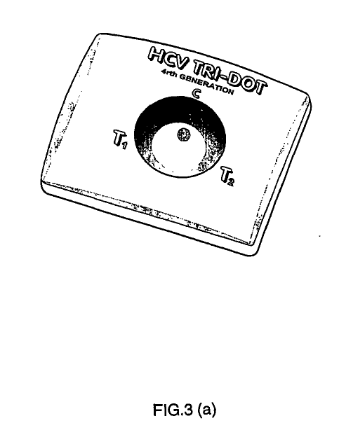

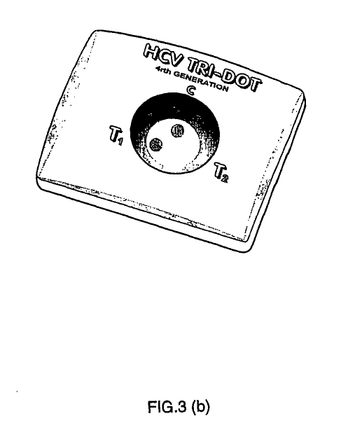

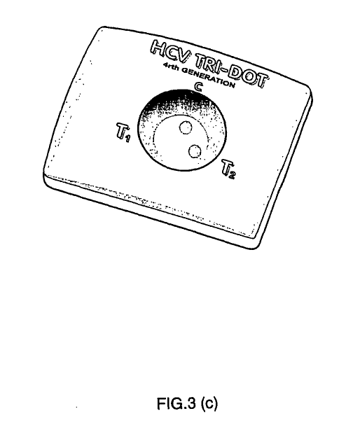

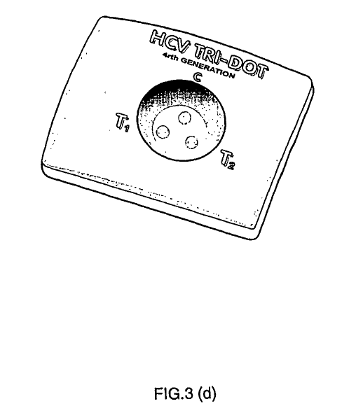

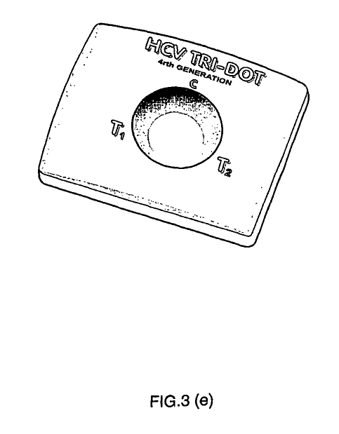

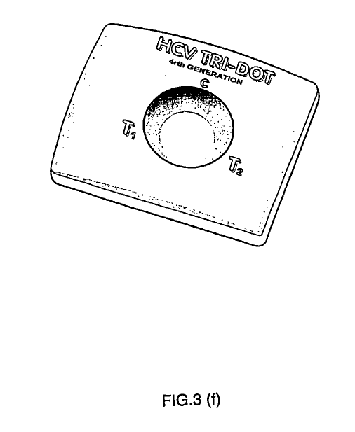

Appearance of only one dot at the control region “C” as shown in FIG. 3(a), indicates that the sample is Non-reactive for antibodies to HCV. Appearance of two dots as shown in FIG. 3(b), one at the control region “C” and other at the test region “T1” indicates that the sample is reactive for antibodies to HCV. Appearance of two dots (as shown in FIG. 3(c), one at the control region “C” and other at the test region “T2” indicates that the sample is reactive for antibodies to HCV and appearance of all the three dots as shown in FIG. 3(d), one each at “C”, “T1” and “T2” region indicates that the sample is reactive for antibodies to HCV.

In case of non-appearance of dots after the completion of the test, either with clear background or with complete pinkinsh or purplish background the test indicates error as shown in FIGS. 3(e and f), this may either be due to procedural error or deterioration of specimen/reagents or particulate matter in the specimen.

For the appearance of said dot at the quality in built control dot “C”, the 0.5 microliter of anti-human IgG solution is coated on the said membrane (6).

At the said T1 and T2 dots the 0.5 microliter of HCV Peptides and Recombinant HCV antigen solution is poured for the indication of dots as hereinbefore mentioned.

The HCV antigens are immobilized on a porous immunofilteration membrane. The sample and the reagents passes through the membrane and are absorbed into the underlying absorbent pad. As the patient's sample passes through the membrane, HCV antibodies if present in serum/plasma, binds to the immobilized antigens and in the subsequent washing step unbound serum or plasma proteins are removed.

The Protein A conjugate is added which binds the Fc portion of the HCV antibodies to give distinct colour near the test region. At the control region the built in quality control Dot confirms the proper functioning of the device, reagent and correct procedural application.

Antibodies present therein corresponds to the different types of antigens as Core; NS3, NS4 and NS5. In the test sample these antigens are added in a specific ratio percentage. The ratio % age of all the antigens is to be proportionally matched keeping in view the ratio of blood or sample deposited thereon. The NS5 antigen have been found to indicate the presence of HCV antibodies at the very initial stages of the infection.

The buffer solution used in the subject invention is selected form the phosphate group mixed with surfactant, stabilizer, sodium chloride and preservative.

The buffer are selected from the group consisting of Disodium Hydrogen Phosphate and Sodium Dihydrogen Phosphate having molar ratio of 8-12 millimolar each.

The stabilizer used in the subject buffer solution is a Protein Stabilizer preferably Bovine serum Albumin.

The surfactant may be selected from the non-ionic, anionic and Zwitterionic surfactant, preferably the surfactant used is Non-ionic surfactant.

The preservative used in the subject buffer solution is selected from Thiomerosal, Sodium Azide and the like, preferably the preservative used is sodium Azide.

The subject buffers solution comprises:

| | |
|---|---|
| Disodium Hydrogen Phosphate | 8-12 millimolar; |
| Sodium Dihydrogen Phosphate | 8-12 millimolar; |
| Surfactant | 1-2% by volume |
| Protein Stabilizer | 0.5-1.5% |
| Sodium Chloride | 0.6-1.5% |
| and preservative | 0.001-0.15% |
| and the balance being distilled water | |

In the process for the preparation of such buffer solution, Disodium Hydrogen Phosphate and Sodium Dihydrogen Phosphate are mixed in the distilled water to get a solution. To this solution is added protein stabilizer and nonionic surfactant in the ratio of 0.5-1.5% and 1-2% each respectively.

Sodium Chloride is then added in the said solution followed by the addition of preservative in the said resultant solution. The pH of the said resultant solution is adjusted from 7-8.5.

The resultant solution is stirred to get the homogenous solution. The said homogenous solution is then filtered in a 0.1-0.22 micron anti-Bactedal filter to get the Buffer Solution of the subject invention to be used in the diagnostic kit for the detection of antibodies of Hepatitis C-virus.

The subject solution is stored at a temperature of 2-8C having a shelf life of about two to two and half years.

The Protein-A conjugate solution of the subject invention comprises Colloidal Gold solution, Protein-A and stabilizers.

The colloidal gold having optical density between 0.8 to 10 is prepared by adding Auric chloride in the distilled water in the ratio of 1 gm/100 ml. The said solution is then boiled and in the said solution sodium citrate solution is added. The solutions comprising Auric Chloride solution and Sodium Citrate solution are boiled till the solution changes its colour. The boiled solution is then allowed to cool at room temperature in the clean environment to get the colloidal gold solution.

The optical density of the said concentrated colloidal gold solution is maintained at 2.5 to 3.5 at wavelength of from 515-530.

The said Sodium Citrate solution is prepared by mixing 1 gm. of Sodium Citrate in 100 ml of distilled water.

The Protein-A is then added to the said colloidal gold solution by vigorously stirring the same for at least half an hour. The stirring is done at the rate of 500-600 RPM. The protein-A attaches to the surface of said colloidal gold spheres of the said colloidal gold solution to get the protein-A conjugate. The pH of the said protein-A conjugate solution is maintained at 5.5-8.0. The said mixing is done at the room temperature.

To the said protein-A conjugate solution, stabilizers are added to block the unblocked sites of the said colloidal gold particles. The said conjugate solution having colloidal gold particles and stabilizers are kept at room temperature for at least half an hour to 24 hours.

The optical density of the said solution is then measured to be at 510-530 nanometer.

The said conjugate solution is then centrifuged at a very high speed of 10,000-15,000 RPM at a temperature of 2-10 degrees to allow the colloidal gold to settle down and the supernatant solution is then decanted by vacuum or any other conventional means. The colloidal gold which is in the pellet form settled at the bottom is suspended in a Buffer solution having pH at 6-8.

In Protein-A conjugate composition comprises 1 ml. of colloidal gold solution having colloidal gold of particle size 18-25 nannometer; 20 ml. of Protein A to be adhered on to the said colloidal gold particles to form Protein A conjugate; a stabilizer to block the unblocked sites of said Protein A Conjugate and Buffer solution for the suspension of said Protein A conjugate.

The Buffer solution used in the subject invention for the suspension of Protein-A conjugate comprises buffers selected from the phosphate group mixed with stabilizer and preservative.

The buffer are selected from the group consisting of Disodium Hydrogen Phosphate and Sodium Dihydrogen Phosphate having molar ratio of 10 millimolar each.

The stabilizer used in the subject buffer solution is a Protein Stabilizer preferably Bovine serum Albumin.

The preservative used in the subject buffer solution is selected from Thiomerosal, Sodium Azide and the like, preferably the preservative used is sodium Azide.

The subject buffers solution comprises:

| | |
|---|---|
| Disodium Hydrogen Phosphate | 10-100 millimolar; |
| Sodium Dihydrogen Phosphate | 10-100 millimolar; |
| Protein Stabilizer | 0.01-2% |
| Preservative | 0.001-0.15% |
| and the balance being distilled water. | |

In the process for the preparation of such buffer solution, Disodium Hydrogen Phosphate and Sodium Dihydrogen Phosphate are mixed in the distilled water to get a solution. To this solution is added protein stabilizer followed by the addition of preservative in the said resultant solution. The pH of the said resultant solution is adjusted from 6-8.5.

The resultant solution is stirred to get a homogenous solution. The said homogenous solution is then filtered in a 0.1 to 0.5 micron anti-Bacterial filter to get the Buffer Solution of the subject invention to be used in for the suspension of colloidal gold for the diagnostic kit for the detection of antibodies of Hepatitis C-virus.

The kit of the subject application may be stored at 4-8 degrees C. in the driest area available. The shelf life of the kit is 12 months from the date of manufacturing.

The kit of the subject application has successfully resulted in providing the end results within 3-5 minutes.

The subject application is a mere statement of invention, where various improvements and alternations are possible without deviating from the scope of the invention, hence the same should not be construed to restrict the scope of the invention.

We claim:

1 A rapid, visual, sensitive and qualitative in vitro diagnostic kit for the detection of antibodies of Hepatitis C Virus in human serum and plasma comprising a testing device, a buffer solution and a Protein A Conjugate, wherein said testing device comprises a base, an absorbent pad made up of cellulosic material having a thickness preferably of 2.4 to 2.7 mm positioned on the said base, an immunofilteration membrane made up of cellulosic material having a pore size of 0.8-1.5 micron and diameter of 12 mm having at least three coatings of a homogenous mixture of different HCV recombinant antigens and HCV Peptides and the antihuman IgG solution for the detection of HCV antibodies mounted over said absorbent pad disposed on the said base, a top cover fitting tightly and removably attached to said base having a central hole conforming to the circumference of said immunofilteration membrane provided with two test dots and one built in quality control dot within the circumference of said immunofilteration membrane characterized in that the invitro diagnostic kit renders 100% sensitivity and 98.9% specificity of the sample under test.

2 The invitro diagnostic kit as claimed in claim 1, wherein the antigens are HCV core antigens, HCV NS3 antigens, HCV NS4 antigens, and HCV NS5 antigens.

3 The invitro diagnostic kit as claimed in claim 1, wherein the antigens are 40-60 nanograms HCV core antigens, 100-150 nanograms HCV NS3 antigens, 150-200 nanograms HCV NS4 antigens, and 150-200 nanograms HCV NS5 antigens.

4 The invitro diagnostic kit as claimed in claim 1, wherein the homogenous mixture is obtained by thoroughly mixing the antigens with a dispensing media.

5 The invitro diagnostic kit as claimed in claim 1, wherein the immunofilteration membrane has the coating of homogenous mixture.

6 The invitro diagnostic kit as claimed in claim 1, wherein HCV Peptides or recombinant HCV antigens are coated on the said membrane at the said T1 and T2 dots.

7 The invitro diagnostic kit as claimed in claim 1, wherein the IgG solution is coated on the said membrane at the said built in quality control dot “C”.

8 The invitro diagnostic kit as claimed in claim 4, wherein said dispensing media is a buffer solution.

9 The invitro diagnostic kit as claimed in claim 8, wherein said dispensing buffer solution comprises 10-100 millimolar Disodium Hydrogen Phosphate, 10-100 millimolar Sodium Dihydrogen Phosphate, 0.1-5% Protein Stabilizer, 0.022-1% Detergent, 10-20% Glycerol, 0.001-0.15% preservative and distilled water.

10 The invitro diagnostic kit as claimed in claim 9, wherein said detergent is selected from Dithiothreitol, t-octyl phenoxy poly ethoxy ethanol, Poly oxy ethylene sorbitan monolaurate, 3-(3-Cholamidopropyl) dimethyl ammonio]-1Propane sulfonate.

11 The invitro diagnostic kit as claimed in claim 1, wherein the said buffers solution comprises 8-12 millimolar Disodium Hydrogen Phosphate, 8-12 millimolar Sodium Dihydrogen Phosphate, 1-2% Surfactant by volume, 0.51.5% Protein Stabilizer, 0.6-1.5% Sodium Chloride, 0.001-0.15% preservative and the distilled water.

12 The invitro diagnostic kit as claimed in claim 1, wherein the said conjugate solution comprises colloidal Gold Solution, Protein A Conjugate, stabilizer and the buffer solution.

13 The invitro diagnostic kit as claimed in claim 12, wherein the said buffers solution comprises 10-100 millimolar Disodium Hydrogen Phosphate, 10-100 millimolar Sodium Dihydrogen Phosphate, 0.01-2% Protein Stabilizer, 0.001-0.015% preservative and the distilled water.
